(19) United States
(12) Patent Application Publication
(10) Pub. No.: US 2024/0225474 A1
(43) Pub. Date: Jul. 11, 2024

(54) SYSTEMS AND METHODS FOR IDENTIFYING AND LOCATING MARKERS USING RFID AND ORTHOGONAL SEQUENCES

(71) Applicant: Cianna Medical, Inc., Aliso Viejo, CA (US)

(72) Inventors: John E. Greene, Valley Center, CA (US); Nikolai Rulkov, San Diego, CA (US); Eduardo Chi Sing, Dana Point, CA (US)

(21) Appl. No.: 18/404,046

(22) Filed: Jan. 4, 2024

Related U.S. Application Data
(60) Provisional application No. 63/478,542, filed on Jan. 5, 2023.

(51) Int. Cl. A61B 5/06; A61B 90/00; G06K 19/07; G06K 19/077

(52) U.S. Cl. CPC A61B 5/064; A61B 90/39; G06K 19/0708; G06K 19/07771; G06K 19/07779; A61B 2090/3979

(57) ABSTRACT

Systems and methods are provided for identifying and locating a plurality of RFID markers implanted within a target tissue region within a patient's body. A probe is provided that may receive signals from the RFID markers. The RFID markers modulate signals from the respective markers based on code sequences opening and closing respective switches of the markers to modulate the backscatter signals. The probe processes the signals to separate the signals based at least in part on the code sequences to identify each of the plurality of markers.

### RELATED APPLICATION

This application claims priority to U.S. Provisional Application No. 63/478,542, filed on Jan. 5, 2023, and titled “SYSTEMS AND METHODS FOR IDENTIFYING AND LOCATING MARKERS USING RFID AND ORTHOGONAL SEQUENCES” which is hereby incorporated by reference in its entirety.

### TECHNICAL FIELD

The present disclosure relates to implantable tags or markers and to systems and methods for identifying and/or locating multiple markers within a patient's body, e.g., during surgical procedures or other procedures, such as during lumpectomy procedures.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 shows an exemplary embodiment of a system including a probe for identifying and/or locating a plurality of reflectors, tags, or markers that may be implanted within a patient's body.

FIGS. 2A-2C are top, side, and end views, respectively, of an exemplary embodiment of a marker for implantation within a patient's body.

FIG. 3 is a schematic showing an exemplary system including an RFID marker implantable within a patient's body and a probe or reader transmitting RF energy and establishing communication with the marker.

FIG. 4 is a schematic showing an exemplary embodiment of a system including an implantable marker including circuitry, powered and/or modulated by RF energy and light energy, and a probe showing representative signal processing by the reader to acquire data from the backscatter signal from the marker.

FIG. 5 is a block diagram of an orthogonal code circuit to control a switch of a marker in order to generate a code sequence that is orthogonal to other markers.

FIG. 6A is a side view of an exemplary embodiment of a probe localizing a plurality of markers 40′ implanted within a breast using RFID.

FIG. 6B is a side view of an exemplary embodiment of a probe localizing a plurality of markers implanted within a breast using RFID and IR light.

FIG. 7A illustrates an exemplary set of orthogonal periodic code sequences that may be used to switch a plurality of markers triggered by RF modulation.

FIG. 7B illustrates an exemplary set of orthogonal periodic code sequences that may be used to switch a plurality of markers triggered by IR pulses.

FIG. 8 illustrates an exemplary set of orthogonal code sequences using a balanced Gold Code that may be used to switch a plurality of markers.

FIG. 9 is a graph showing the cross-correlation resulting from using the orthogonal balanced Gold Code sequences shown in FIG. 8.

### DETAILED DESCRIPTION

Before a biopsy or surgical procedure to remove a lesion within a breast, e.g., during a lumpectomy procedure, the location of the lesion must be identified. For example, mammography or ultrasound imaging may be used to identify and/or confirm the location of the lesion before the procedure. The resulting images may be used by a surgeon during the procedure to identify the location of the lesion and guide the surgeon, e.g., during dissection to access and/or remove the lesion. However, such images are generally two dimensional and therefore provide only limited guidance for localization of the lesion since the breast and any lesion to be removed are three-dimensional structures. Further, such images may provide only limited guidance in determining a proper margin around the lesion, i.e., defining a desired specimen volume to be removed.

To facilitate localization, immediately before a procedure, a wire may be inserted into the breast, e.g., via a needle, such that a tip of the wire is positioned at the location of the lesion. Once the wire is positioned, it may be secured in place, e.g., using a bandage or tape applied to the patient's skin where the wire emerges from the breast. With the wire placed and secured in position, the patient may proceed to surgery, e.g., to have a biopsy or lumpectomy performed.

One problem with using a wire for localization is that the wire may move between the time of placement and the surgical procedure. For example, if the wire is not secured sufficiently, the wire may move relative to the tract used to access the lesion and consequently the tip may misrepresent the location of the lesion. If this occurs, when the location is accessed and tissue removed, the lesion may not be fully removed and/or healthy tissue may be unnecessarily removed. In addition, during the procedure, the surgeon may merely estimate the location of the wire tip and lesion, e.g., based on mammograms or other images obtained during wire placement, and may proceed with dissection without any further guidance. Again, since such images are two dimensional, they may provide limited guidance to localize the lesion being treated or removed.

Alternatively, it has been suggested to place a radioactive seed to provide localization during a procedure. For example, a needle may be introduced through a breast into a lesion, and then a seed may be deployed from the needle. The needle may be withdrawn, and the position of the seed may be confirmed using mammography. During a subsequent surgical procedure, a hand-held gamma probe may be placed over the breast to identify a location overlying the seed. An incision may be made and the probe may be used to guide excision of the seed and lesion.

Because the seed is delivered through a needle that is immediately removed, there is risk that the seed may migrate within the patient's body between the time of placement and the surgical procedure. Thus, similar to using a localization wire, the seed may not accurately identify the location of the lesion, particularly, since there is no external way to stabilize the seed once placed. Further, such gamma probes may not provide desired precision in identifying the location of the seed, e.g., in three dimensions, and therefore may only provide limited guidance in localizing a lesion.

Accordingly, apparatus and methods for localization of lesions or other tissue structures in advance of and/or during surgical, diagnostic, or other medical procedures would be useful.

Embodiments herein are directed to implantable tags or markers, and to systems and methods for identifying and/or locating multiple markers within a patient's body, e.g., during surgical procedures or other procedures, such as during lumpectomy procedures.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

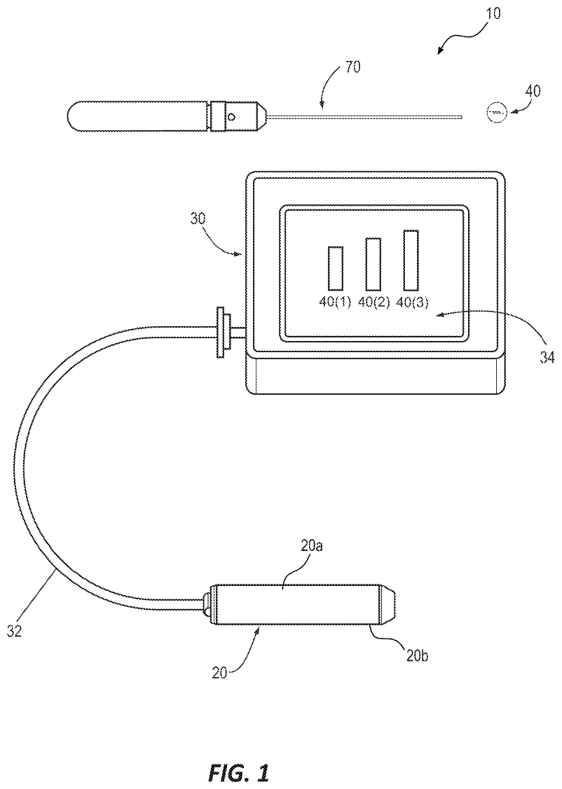

Turning to the drawings, FIG. 1 shows an exemplary embodiment of a system 10 for localization of a target tissue region within a patient's body that includes a probe 20 and a plurality of tags or markers 40 that may be implanted within a patient's body, e.g., within a target tissue region, such as within a breast. In some embodiments, the markers 40 may comprise passive Radio-frequency identification (RFID) tags or active RFID tags.

In some embodiments, the system 10 may include one or more additional components, e.g., one or more delivery devices 70, each carrying one or more reflectors, tags, or markers 40 (one shown) for introduction/implantation in a patient's body, and a controller and/or display unit 30 coupled to the probe 20, e.g., using one or more cables 32, similar to embodiments described in the applications incorporated by reference herein.

The probe 20 may include one or more antennas for transmitting RFID electromagnetic signals into a patient's body and receiving reflected signals from the patient's body. The antenna(s) may transmit a radio frequency for simultaneous detection of the markers 40 based on modulated radio-frequency properties. For example, a switch inside each marker 40 may be used for changes in load manipulation or backscatter coupling. In some embodiments, the antenna(s) may comprise a transmit and receive antenna. In other embodiments, the antenna(s) may feature a single antenna to transmit and receive.

The probe 20 may be a portable device having electromagnetic signal emitting and receiving capabilities, e.g., a RFID probe. The probe 20 may be a handheld device including a first or proximal end 20a configured to be held by a user, and a second or distal end 20b configured to be placed against or adjacent tissue, e.g., a patient's skin or underlying tissue.

The probe 20 may include one or more processors within its housing or within the display unit 30 including one or more controllers, circuits, signal generators, gates, and the like (not shown) needed to generate signals for transmission by transmit antennas and/or to process signals received from receive antennas. The components of the processor(s) may include discrete components, solid state devices, programmable devices, software components, and the like, as desired. Optionally, the probe 20 and/or display unit 30 may include other features or components, such as one or more user interfaces, memory, transmitters, receivers, connectors, cables, power sources, and the like (not shown). In addition, the processor(s) may be coupled to a display 34 of the display unit 30 for displaying information to a user of the probe 20, e.g., spatial or image data obtained using the probe 20.

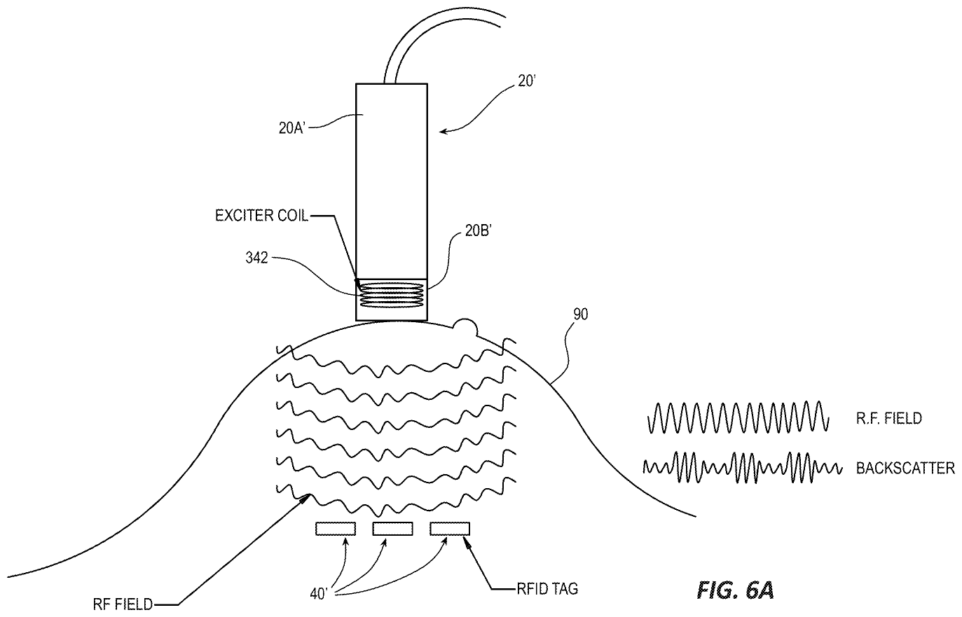

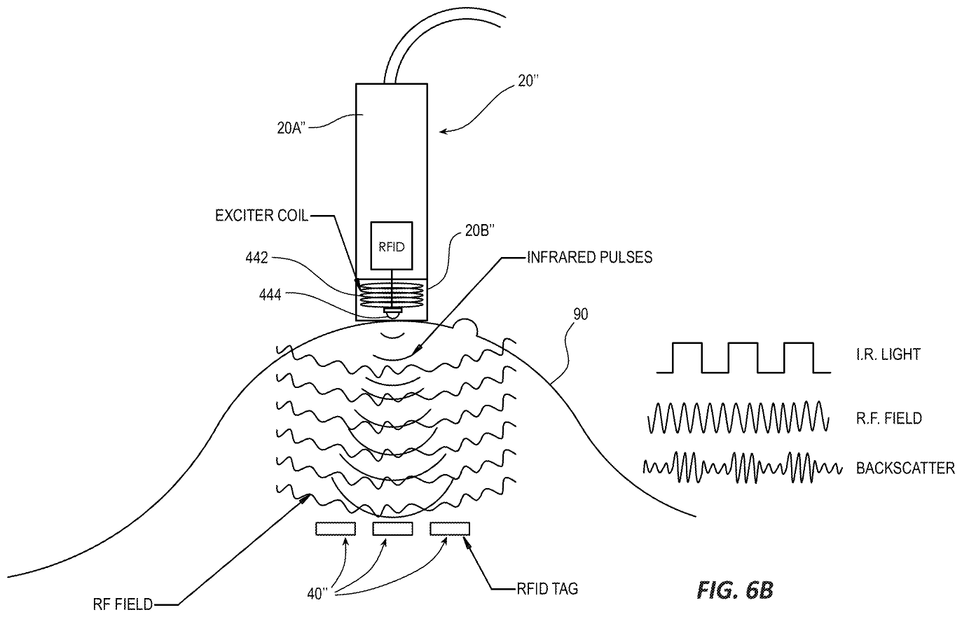

The system 10 may be used during a medical procedure, to identify and locate a plurality of tags or markers 40 implanted within a patient's body. For example, in a breast biopsy or lumpectomy procedure, the markers 40 may be used to facilitate localization of a lesion or other target tissue region and/or to facilitate dissection and/or removal of a specimen from a breast 90, as shown in FIG. 6A and FIG. 6B. It should be noted that, although the system may also be used in localization of other objects in other areas of the body

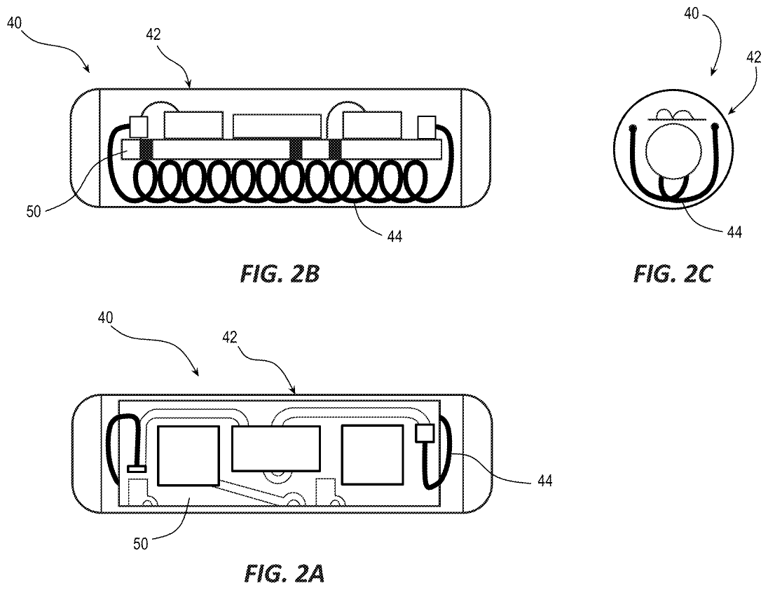

FIGS. 2A-2C show an exemplary embodiment of a marker 40 that may be used for each of the markers that may be implanted within a patient's body. Generally, the marker 40 includes an electronics package 42 coupled to one or more antennas 44. In an exemplary embodiment, the antenna 44 may be a coil with a matching network. The antenna 44 may also be contained within the package 42, e.g., on the base, or may be located at least partially outside the package 42. In some embodiments, the antenna may be an antenna coil on the substrate 50.

The marker 40 may include one or more circuits or other electrical components encased or embedded in the electronics package 42 and configured to perform load modulation or backscatter modulation to provide a probe 20 with identifying information. Further, based on the strength of the coupling between the probe 20 and the marker 40, a distance between the probe 20 and the marker may be determined. In some embodiments, the components may be mounted on a semiconductor chip, print circuit board (PCB), and/or other substrate 50 carried in the package 42, and encased within the package 42 such that the components are electrically isolated from one another.

The electronics package 42 may include circuitry to open and close a switch that is coupled in parallel to the antenna 44 to change the load properties or backscatter coupling of the marker 40. The marker may use either load modulation or backscatter modulation to send information to the probe. For example, when the marker 40 desires to send a logic ‘0’, the electronics package 42 may open the switch parallel the antenna 44 to match a transmitting antenna on a probe in impedance achieving maximum power transfer. This can be detected by the probe as a drop in reflected voltage level. When the marker 40 desires to send a logic ‘1’, the electronics package 42 may close the switch to cause short across the antenna 44 purposely mismatching the antenna 44 so that the marker 40 emits back all or most of the energy from the transmitting antenna on the probe.

Signaling between the marker 40 and the probe 22 may be done using different techniques. For example, in some embodiments, the marker may use load modulation. The marker 40 may modulate the field produced by the probe 22 by changing the load properties that the marker 40 represents in relation to the probe 22. By switching between lower and higher relative loads, the marker 40 may produce a change that the probe 22 can detect.

In some embodiments, the marker 40 can produce a backscatter signal. The marker 40 may produce the backscatter signal by switching reflective properties of the antenna 44. For instance, a marker 40 may switch between absorbing and reflecting incident waves from the probe 22. Active markers may contain transmitters capable of transmitting a signal without separate from the signal sent from the probe 22.

In some embodiments, the marker 40 may communicate over the RFID interface while being powered by infrared light from the probe 20. For instance, when the infrared light is on, the marker 40 may have sufficient power to modulate a backscatter signal or perform load modulation to communicate an identification to the probe 20. The probe 20 may use the signal strength of the data from the marker 40 to determine a distance between the marker 40 and the probe 20.

In some embodiments, the modulation of the marker 40 may be controlled by the infrared light from the probe 20. For example, the probe 20 may turn the infrared light on and off and the marker 40 may open and close the switch that is parallel with the antenna in sync with the infrared light turning on and off. The strength of the resulting modulation in backscatter or load may be used by the probe 20 to determine a distance between the marker 40 and the probe 20.

In some embodiments, the marker 40 may be configured to multiplex the backscatter signal or the load modulation in a non-interfering pattern. That is, the marker 40 may multiplex the backscatter or load in a way that does not interfere with other markers. In some embodiments, the markers may multiplex the data using orthogonal codes to limit interference.

In some embodiments, electronics package 42 may include circuitry to determine a synchronization signal and a clock signal from the probe 20. The multiplexing may be based on the synchronization signal and the clock signal. The synchronization signal may provide an indication of a beginning of a multiplexing frame so that multiplexing from multiple markers may be aligned.

The probe 20 may transmit the synchronization signal and the clock signal using radio frequency (RF) modulation or an infrared light. The marker 40 may also be powered by either RF or infrared light. The synchronization signal may indicate a beginning of a frame that codes of a marker 40 should be transmitted within. The marker 40 may transmit the code sequence during the frame and use the clock signal for the timing of the transmission.

In some embodiments, the clock signal and/or the synchronization signal may be sent by the probe 22 via RF signal modulation (e.g., phase). In some embodiments, the clock signal and/or the synchronization signal may be sent by the probe 22 via infrared lighting. In some embodiments, a combination of infrared lighting and RF signal modulation may be used. For example, the clock signal may be sent by the probe 22 via RF signal modulation and the synchronization signal may be sent by the probe 22

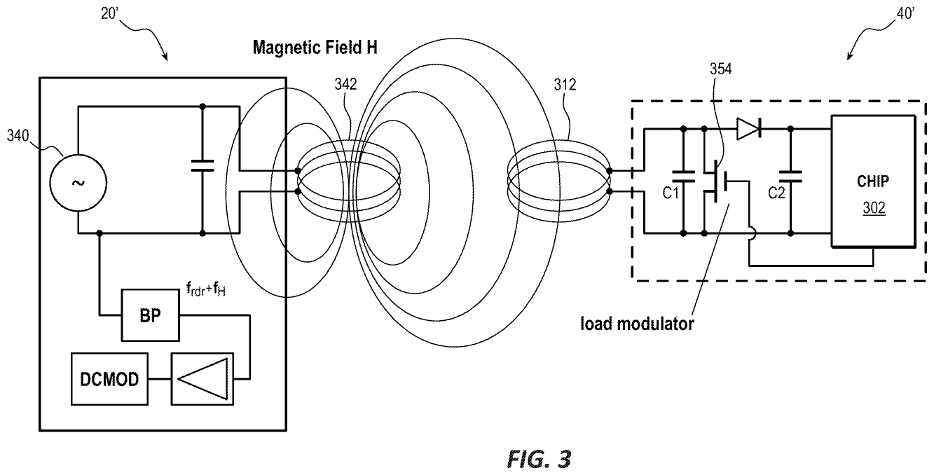

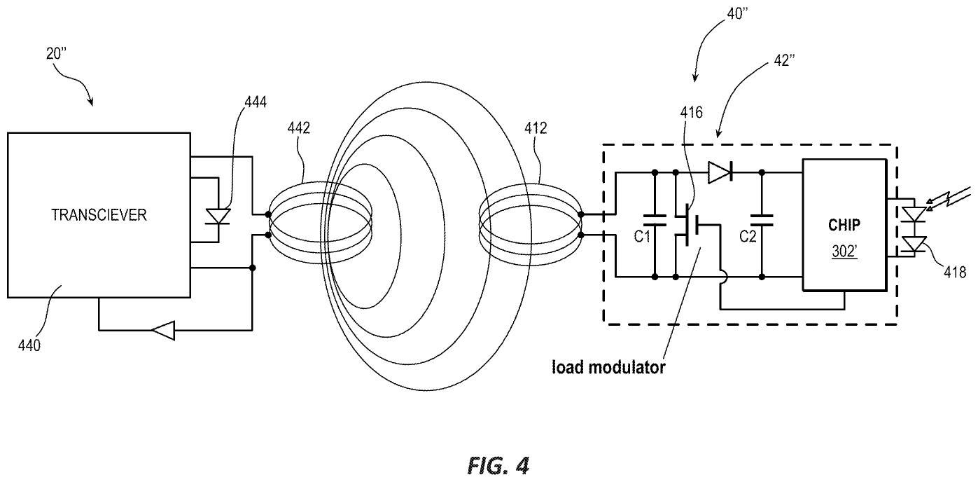

FIGS. 3 and 4 illustrate block diagrams of two embodiments of a probe and a marker using RFID. The embodiments shown in FIGS. 3 and 4 illustrate two potential embodiments for the electronics package 42 of FIGS. 2A-2C. More specifically, FIG. 3 illustrates a probe 20′ and a marker 40′ using RF energy, and FIG. 4 illustrates a probe 20″ and a marker 40″ using RF energy and light.

As shown in FIG. 3, an RFID tag (e.g., marker 40′) may communicate to an interrogator or reader (e.g., probe 20′) via backscatter modulation. The marker 40′ may include an antenna 312, a control circuit 302, and a switch 354. A probe 20′ may include a RF signal generator 340 and an antenna 342 that transmits RF energy and establishes communication with the marker 40′. The marker 40′ may be powered using the RF energy from the probe 20′.

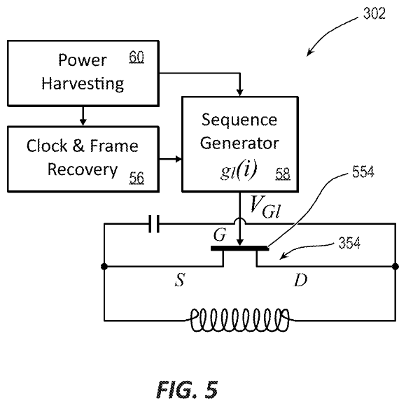

The switch 354 may be a field effect transistor (FET), e.g., a junction field effect transistor (JFET), coupled to the control circuit 302. Shown with more detail in reference to FIG. 5, the control circuit 302 may control the switch in order to generate a code sequence in the backscatter signal. The code sequence may be orthogonal to other markers. In some embodiments, the control circuit 302 may also determine a synchronization signal and/or a clock signal from RF signal modulation transmitted by the probe 20′ (e.g., phase modulation).

The probe 20′ may use the orthogonal code sequences to identify multiple markers. In some embodiments, when the control circuit 302 desires to send a logic ‘0’, the control circuit 302 may open the switch 354. When the control circuit 302 desires to send a logic ‘1’, the control circuit 302 may close the switch 354 to short across the antenna 312, altering the antennas 312 reflective properties or the load properties of the marker 40′

As described further elsewhere herein, the control circuit 302 of each marker 40′ may be pre-programmed such that the code sequences generated by the control circuit 302 are orthogonal to one another, i.e., the control circuit 302 may open and close the respective switches 354, to modulate the reflective properties of the markers 40′ differently from one another, and the probe 20′ may be configured to analyze the backscatter signals or load modulation to identify and locate each of the markers 40′ substantially simultaneously based on the resulting modulation in the backscatter signals received by the probe 20′ or the load modulation.

In some embodiments, the orthogonal codes may be used by the markers 40′ to multiplex each symbol of the return data from the markers 40′. In other words, the orthogonal codes can be used to encode each of the return data symbols to further the functionality of the markers 40′. For example, the markers 40′ may use different code sequences (e.g., Barker codes) to represent different symbols. For example, eight Barker codes could represent a symbol of three bits. Embodiments using the orthogonal codes to multiplex symbols of the return data may not need to synchronize the frames of all the markers 40′. For example, the probe 20′ may send the received data into a tapped register and when the correct number of bits has been shifted, the summed output may show correlation with a particular code.

A processor of the probe 20′ may process the modulated backscatter signals received by the antenna 342 or the load modulation to determine information regarding the marker 40′. The probe 20′ may use a band pass filter and a demodulator to process the modulated backscatter. The probe 20′ may use the amplitude of the backscatter signals to determine the distance from the distal end of the probe 20′ to the marker 40′ (i.e., the “range”). In some embodiments, the marker 40′ may modulate the backscatter signals to provide a marker identification to the probe 20′. In some embodiments, the marker 40′ may include additional information in the backscatter signals.

In some embodiments, the transmitter coil antenna 342 of the probe 20′ may transmit a uniform sine wave, e.g., at a frequency between about one hundred twenty kilohertz and two thousand four hundred fifty Megahertz (120 KHz-2450 MHz) depending on the RFID band being used, e.g., LF (125-134.2 KHz), HF (13.56 MHZ), UHF (860-960 MHZ), or SHF (2450 MHZ). The transistor of the marker 40′ may alternately, e.g., periodically, short the antenna of the marker 40′, which may modulate the backscatter signals transmitted by the marker 40′. Optionally, synchronous demodulation techniques may be utilized to further improve receiver sensitivity at the reader antenna 342.

In some embodiments, the probe 20′ may localize a plurality of markers 40′ implanted within a breast using RFID as described with reference to FIG. 6A.

FIG. 4 illustrates a block diagram of an exemplary embodiment of an RFID marker 40″ that uses light energy rather than or in addition to RF energy, to power the marker 40″. The probe 20″ uses RF energy for detecting backscatter level and modulation. Such a configuration may optimize the marker 40″ for maximum range and consistency.

The marker 40″ may include an antenna 412, a control circuit 302′, a switch 416 (e.g., a MOSFET transistor), and one or more photodiodes 418. In the illustrated embodiment, the control circuit 302′ is coupled to the switch 416 to cause the switch to open and close. The control circuit 302′ may operate the switch 416 to create an identification code within the backscatter or load modulation. The code may be orthogonal to other markers codes to limit interference. The control circuit 302′ may also be coupled to photodiode(s) 418 to harvest power from infrared light emitted by the probe 20″. In some embodiments, the control circuit 302′ may also determine a synchronization signal and/or a clock signal from the infrared light.

In an exemplary embodiment, a plurality of photodiodes 418 may be connected in series, and capable of transforming incident light striking them into electrical energy. Optionally, the photodiodes 418 may be provided in multiple pairs connected in series, which may be arranged at multiple different positions relative to the package (not shown) to ensure at least one of the photodiodes is aligned properly to receive the infrared light. The package of the marker 40″ may be at least partially transparent or the photodiodes 418 may be exposed from the package such that light directed towards the package may be received by the photodiodes 418.

In an exemplary embodiment, the photodiode(s) 418 may be configured to convert infrared light to electrical energy. One advantage of infrared energy is that it may pass sufficiently through tissue such that the probe 20″, when placed against a patient's skin, may deliver sufficient energy to activate a relatively small marker 40″, when implanted several inches away within the patient's body, e.g., within a breast. Optionally, the photodiode(s) 418 (and/or a transparent surface of the package overlying the photodiode(s) 418) may include one or more coatings and/or filters, e.g., to narrow the band of infrared light striking the photodiode(s) 418, to distinguish different tags, and the like.

Shown with more detail in reference to FIG. 5, the control circuit 302′ may operate the switch in order to generate a code sequence. In some embodiments, the code sequence may be orthogonal to other markers. The probe 20″ may use the code sequence to identify the marker 40″.

In some embodiments, when the orthogonal code circuit 302′ desires to send a logic ‘0’, the control circuit 302′ may open the switch 416. When the control circuit 302′ desires to send a logic ‘1’, the orthogonal code circuit 302′ may close the switch 416 to short across the antenna 412.

As described further elsewhere herein, the control circuit 302′ of each marker 40″ may be pre-programmed such that the code sequences generated by the control circuit 302′ are orthogonal to one another, i.e., the control circuit 302′ may open and close the respective switches 416, to modulate the reflective properties of the markers 40″ differently from one another, and the probe 20″ may be configured to analyze the reflected signals to identify and determine a distance to each of the markers 40″ substantially simultaneously based on the resulting modulation in the backscatter signals received by the probe 20″.

In some embodiments, the orthogonal codes may be used by the markers 40′ to multiplex each symbol of the return data from the markers 40′. In other words, the orthogonal codes can be used to encode each of the return data symbols to further the functionality of the markers 40′. For example, the markers 40′ may use different code sequences (e.g., Barker codes) to represent different symbols. For example, eight Barker codes could represent a symbol of three bits. Embodiments using the orthogonal codes to multiplex symbols of the return data may not need to synchronize the frames of all the markers 40′. For example, the probe 20′ may send the received data into a tapped register and when the correct number of bits has been shifted, the summed output may show correlation with a particular code.

The control circuit 302′ may be coupled to the photodiodes 418 such that the infrared pulses may generate electrical energy, which may be harvested by the circuit. The control circuit 302′ may store sufficient energy to activate the switch (i.e., selectively closing the switch 416) to include information in the backscatter signals transmitted by the antenna 412.

For example, in this embodiment, by alternating between two modes such as ‘information’ mode and ‘distance’ mode, synchronous detection may be used to identify, determine a distance, and/or communicate with the marker 40″. In an exemplary embodiment, the control circuit 302′ may include an orthogonal code that may be transmitted in the backscatter signals, to distinguish the tag from other tags that may also be implanted within a patient's body. In addition or alternatively, the control circuit 302′ may be able to include other data or information, e.g., preprogrammed into the marker 40″, in the signals transmitted back to the probe 20″.

The probe 20″ may be a handheld device that includes a housing including a first or proximal end configured to be held by a user, and a second or distal end intended to be placed against or adjacent tissue, e.g., including a substantially flat or other contact surface. The probe 20″ may include a transceiver module 440 including an exciter coil or other antenna 442, and a controller (not shown), one or more processors, one or more output devices, which may be located within the housing and/or in an external control unit.

In addition, unlike the embodiment shown in FIG. 3, the probe 20″ includes a light transmitter 444. The light transmitter may include one or more LEDs, light fibers, and the like, configured to transmit light, e.g., infrared light, into tissue contacted by the probe 20″. For example, light fibers terminating at the distal end of the probe 20″ may extend through a cable to the control unit, such that light from one or more LEDs passes through the light fibers distally to the distal end of the probe 20″. Optionally, the light fibers may include one or more lenses, filters, and the like (not shown), if desired, for example, to focus the light transmitted by the probe 20″ in a desired manner (e.g., in a relatively narrow beam extending substantially parallel to a central axis of the probe 20″, in a wider beam, and the like). Alternatively, the light source, e.g., one or more LEDs, may be located within the probe 20″ e.g., adjacent the distal end, and one or more lenses, filters, or other components (not shown) may be coupled to the light source to direct light from the distal end of the probe 20″. In an exemplary embodiment, the light source is an infrared light source, e.g., capable of delivering near infrared light between, for example, eight hundred and nine hundred fifty nanometers (800-950 nm) wavelength.

During operation, the probe 20″ may simultaneously transmit radiofrequency signals and optical signals, which may be received by the marker 40″. In response, the optical signals energy may modulate backscatter signals transmitted by the marker 40″ and/or may be used to power the marker 40″. The processor of the probe 20″ may process the modulated backscatter signals received by the antenna 442 to determine information regarding the marker 40″. For example, the probe 20″ may use the amplitude of the backscatter signals to determine the distance from the distal end of the probe 20″ to the marker 40″ (i.e., the “range”). In some embodiments, the marker 40″ may modulate the backscatter signals to include information (e.g., a marker identification), which may be extracted from the backscatter signals.

In some embodiments, a clock signal and/or a synchronization signal may be sent by the probe 20″ via RF signal modulation (e.g., phase). In some embodiments, the clock signal and/or the synchronization signal may be sent by the probe 20″ via infrared lighting.

In some embodiments, the transmitter coil antenna 442 of the probe 20″ may transmit a uniform sine wave, e.g., at a frequency between about one hundred twenty kilohertz and two thousand four hundred fifty Megahertz (120 KHz-2450 MHZ) depending on the RFID band being used, e.g., LF (125-134.2 KHz), HF (13.56 MHZ), UHF (860-960 MHz), or SHF (2450 MHZ). Simultaneously, the light transmitter 444 may transmit infrared pulses, e.g., at a frequency between about two hundred Hertz and fifty kilohertz (200 Hz-50 kHz) or between about two hundred Hertz and ten kilohertz (200 Hz-10 kHz), both of which may be received by the marker 40″. The transistor may alternately, e.g., periodically, short the antenna of the marker 40″, which may modulate the backscatter signals transmitted by the marker 40″. Optionally, in this embodiment, synchronous demodulation techniques may be utilized to further improve receiver sensitivity at the reader antenna 442.

In some embodiments, the probe 20″ may localize a plurality of markers 40″ implanted within a breast using RFID as described with reference to FIG. 6B.

FIG. 5 illustrates a block diagram of a control circuit 302 that may be used to open and close a switch 554 to cause a marker to transmit an orthogonal sequence. In the illustrated embodiment, the control circuit 302 may include a clock circuit or block 56, a power harvesting circuit or block 60, and a sequence generator 58. The sequence generator 58 may be coupled to the clock circuit 56 and the switch 554. The control circuit 302 may be used to control a switch 554.

The switch 554 of FIG. 5 is illustrative of the switches 416 and 354 shown in FIGS. 3 and 4. The clock circuit or block 56, a power harvesting circuit or block 60, and a sequence generator 58 may be used in combination with the markers shown in FIGS. 3 and 4.

The clock circuit 56 and the power harvesting circuit 60 may be connected with and receive power from an energy converter. The energy converter may be a RFID antenna (e.g., coil and capacitor) of an RFID marker, or one or more photodiodes, or a combination of the RFID antenna and one or more photodiodes. The power harvesting circuit 60 may receive and store electrical energy to operate one or more electrical components of the marker, such as the sequence generator 58. The switch 554 may be a field effect transistor (FET), e.g., a junction field effect transistor (JFET). Opening and closing the switch may be used to cause backscatter or load modulation.

In some embodiments, light pulses intermittently striking photodiodes may generate a voltage that may be used by the clock circuit 56 to provide a control signal (also referred to herein as a synchronization signal) that may be used to activate the sequence generator 58 to open and close the switch 54, e.g., based on a pre-programmed code sequence, as described elsewhere herein. In some embodiments, the light pulses may also be used by the probe to provide the marker with a clock signal.

In some embodiments, the control signal (e.g., synchronization signal) may be transmitted via RF signaling. For example, the clock circuit 56 may identify the control signal by detecting a phase modulation. When the clock circuit 56 receives the control signal, the clock circuit may activate the sequence generator 58 to open and close the switch 54, e.g., based on a pre-programmed code sequence, as described elsewhere herein. In some embodiments, the RF signal modulation may also be used by the probe to provide the marker with a clock signal.

The power harvesting block 60 may harvest electrical energy, as needed, from the diodes or RFID antenna to provide voltage and/or other electrical energy to the sequence generator 58 and/or other components of the marker. By being able to change the switch 54 from closed to open, the reflection or load properties of the antenna of the marker may be changed significantly and used by the probe to identify, determine a distance, and/or distinguish the markers within the patient's body.

FIG. 6A is a side view of an exemplary embodiment of a probe 20′ localizing a plurality of markers 40′ implanted within a breast using RFID. The probe 20′ may be a handheld device including a first or proximal end 20A′ configured to be held by a user, and a second or distal end 20B′ configured to be placed against or adjacent tissue, e.g., a patient's skin or underlying tissue. An exciter coil (e.g., antenna 342) may be used to transmit an RF field into the tissue and receive backscatter modulated by the markers 40′.

Before the procedure, a target tissue region, e.g., a tumor or other lesion, may be identified using conventional methods. For example, a lesion (not shown) within a breast 90 may be identified, e.g., using mammography and/or other imaging, and a decision may be made to remove the lesion. A plurality of markers 40′ may be implanted within the breast 90 within or adjacent the target lesion, e.g., using individual delivery devices or successively from a single delivery device (e.g., delivery device 70 of FIG. 1).

Once the markers 40′ are implanted, e.g., as shown in FIG. 6A, the probe 20′ may be activated and/or placed against a patient's skin, e.g., against the breast 90. For example, as shown in FIG. 6A, the distal end 20B′ of the probe 20′ may be placed adjacent or in contact with the patient's skin, e.g., generally above the lesion, and/or otherwise aimed generally towards the lesion and markers 40′, and activated to determine a spatial relationship between the markers 40′ and the distal end 20B′ of the probe 20′, e.g., a distance and/or orientation angle, to facilitate determining a proper direction of dissection for the surgeon. The probe 20′ may take multiple measurements of distance and orientation angle between the markers 40′ and the probe 20′ and use the multiple measurements to determine a location of the markers' (e.g., triangulation).

A display (e.g., display 34 of FIG. 1) may include a readout providing distance, angle, orientation, and/or other data based on predetermined criteria, e.g., based on the relative distance from the markers 40′ to the distal end 20B′ of the probe 20. The distance information may be displayed as a numerical value representing the distance in units of length, such as in inches (in.) or centimeters (cm). For example, as shown in FIG. 1, a bar graph may be presented on the display 34 with the height of each bar corresponding to the distance from the respect markers 40′. Alternatively, the display 34 may present a graphical image (e.g., a two-dimensional or three-dimensional image) depicting the markers 40′, the probe 20′, the distance from the probe 20′ to the markers 40′, and/or a physiological picture of the body part containing the markers 40′ (e.g., the breast).

Tissue may then be dissected, e.g., by creating an incision in the patient's skin and dissecting intervening tissue to a desired depth, e.g., corresponding to a target margin around the lesion is reached. A tissue specimen may be excised or otherwise removed using conventional lumpectomy procedures, e.g., with the markers 40′ remaining within the removed specimen.

FIG. 6B is a side view of an exemplary embodiment of a probe 20″ localizing a plurality of markers 40″ implanted within a breast using RFID and IR light. The probe 20″ may be a handheld device including a first or proximal end 20A″ configured to be held by a user, and a second or distal end 20B″ configured to be placed against or adjacent tissue, e.g., a patient's skin or underlying tissue. An exciter coil (e.g., antenna 442) may be used to transmit an RF field into the tissue and receive backscatter modulated by the markers 40″.

In addition, the probe 20″ includes a light source or light transmitter 444 configured to transmit light pulses into tissue contacted by the distal end 20B″, e.g., into breast tissue 90. An antenna 442 may be used to transmit an RF field into the tissue and receive backscatter modulated by the markers 40″.

Before the procedure, a target tissue region, e.g., a tumor or other lesion, may be identified using conventional methods. For example, a lesion (not shown) within a breast 90 may be identified, e.g., using mammography and/or other imaging, and a decision may be made to remove the lesion. A plurality of markers 40″ may be implanted within the breast 90 within or adjacent the target lesion, e.g., using individual delivery devices or successively from a single delivery device (e.g., delivery device 70 of FIG. 1).

Once the markers 40″ are implanted, e.g., as shown in FIG. 6A, the probe 20″ may be activated and/or placed against a patient's skin, e.g., against the breast 90. For example, as shown in FIG. 6A, the distal end 20B″ of the probe 20″ may be placed adjacent or in contact with the patient's skin, e.g., generally above the lesion, and/or otherwise aimed generally towards the lesion and markers 40″, and activated to determine a spatial relationship between the markers 40″ and the distal end 20B″ of the probe 20″, e.g., a distance and/or orientation angle, to facilitate determining a proper direction of dissection for the surgeon. The probe 20″ may take multiple measurements of distance and orientation angle between the markers 40″ and the probe 20″ and use the multiple measurements to determine a location of the markers” (e.g., triangulation).

A display (e.g., display 34 of FIG. 1) may include a readout providing distance, angle, orientation, and/or other data based on predetermined criteria, e.g., based on the relative distance from the markers 40″ to the distal end 20B″ of the probe 20. The distance information may be displayed as a numerical value representing the distance in units of length, such as in inches (in.) or centimeters (cm). For example, as shown in FIG. 1, a bar graph may be presented on the display 34 with the height of each bar corresponding to the distance from the respect markers 40″. Alternatively, the display 34 may present a graphical image (e.g., a two-dimensional or three-dimensional image) depicting the markers 40″, the probe 20″, the distance from the probe 20″ to the markers 40″, and/or a physiological picture of the body part containing the markers 40″ (e.g., the breast).

Tissue may then be dissected, e.g., by creating an incision in the patient's skin and dissecting intervening tissue to a desired depth, e.g., corresponding to a target margin around the lesion is reached. A tissue specimen may be excised or otherwise removed using conventional lumpectomy procedures, e.g., with the markers 40″ remaining within the removed specimen.

An exemplary method will now be presented describing operation of the system 10 of FIG. 1 during use. Initially, when the probe 20 is activated, the antenna may transmit radio frequency (RF) pulses, which may be reflected by the markers 40, surrounding tissue, and/or otherwise by the patient's body. The antennas receive the reflected signals, which include crosstalk, scattering, noise, and reflections from the implanted markers 40. The received signal may be used as a baseline waveform.

After acquisition of the baseline waveform is completed, the probe 20 may send the markers a control signal (also referred to herein as a synchronization signal) and a clock signal. In some embodiments, light pulses from a light source may be used to send the control signal and the clock signal. In some embodiments, the control signal and clock signal may be sent via RF signal modulation (e.g., phase). In some embodiments, the control signal may be sent via RF signal modulation and the clock signal may be sent via the light source. In some embodiments, the control signal may be sent via the light source and the clock signal may be sent via RF signal modulation.

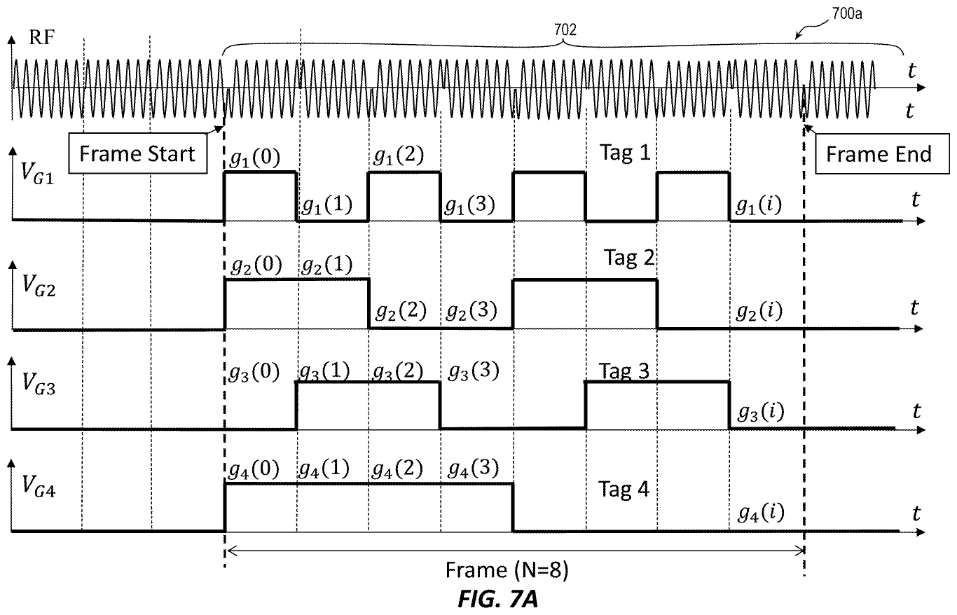

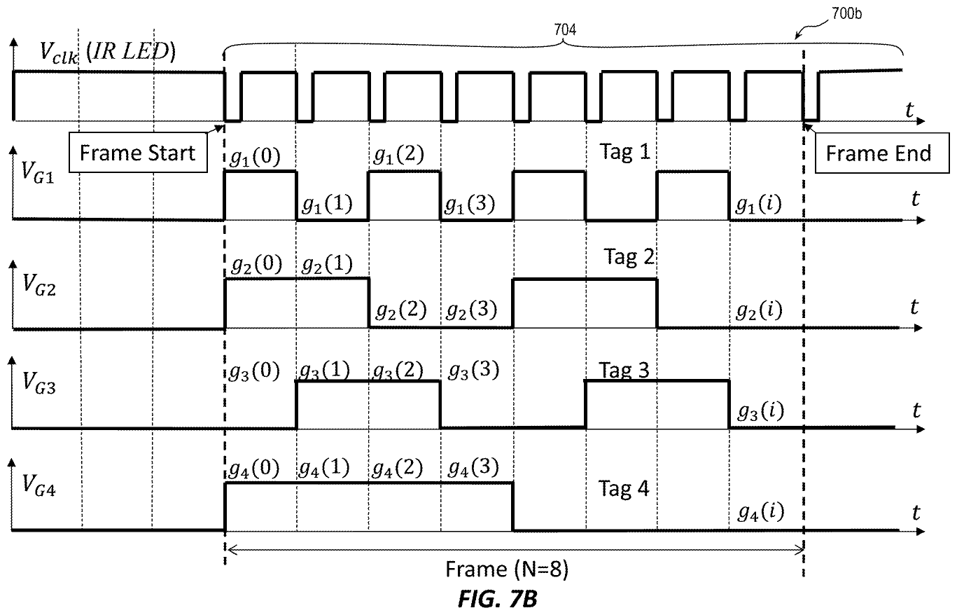

For example, in some embodiments, the light source may be activated to generate a clock pulse. The clock pulse may comprise a plurality of light pulses in spaced-apart frames including a predetermined number of pulses (N) (as shown in FIG. 7B). The light pulses may trigger the change of internal states of the markers 40 in accordance with the preprogrammed code sequence implemented in each marker 40. In response to the light pulses, the clock circuit 56 of each marker 40 may activate the sequence generator 58 to open and close the switch 554 according to the code sequence to short or not short the antennas of each marker by voltage (VGI) at the gate (G) of switch 354 parallel with the antenna, thereby modulating the backscatter signal based on the light pulses.

Similarly, the probe 20 may use RF modulation to generate a clock pulse. The clock pulse may comprise a plurality of phase modulations in the RF signal. The clock pulses may be in spaced-apart frames that include a predetermined number of phase modulations (N) (as shown in FIG. 7A). The RF modulations may trigger the change of internal states of the markers 40 in accordance with the preprogrammed code sequence implemented in each marker 40. In response to the clock pulses, the clock circuit 56 of each marker 40 may activate the sequence generator 58 to open and close the switch 554 according to the code sequence to short or not short the antennas of each marker by voltage (VGI) at the gate (G) of switch 354 parallel with the antenna, thereby modulating the backscatter signal based on the RF modulation.

In some embodiments, the light pulses may power the electrical circuitry of the markers 40 via the diodes 52 and power harvesting block 60 to support the switching sequence. In some embodiments, the RF signal may be used to power the markers 40.

The clock circuit 56 of each marker 40 may process the clock pulses. For example, the clock circuit may detect the changes in voltage output by the diodes 52 when the light pulses strike the diodes or the RF modulation. In some embodiments, the clock circuit 56 may detect clock pulses as the rising edge of the light pulses and framing events encoded as relatively long time intervals with no clocking pulses. In some embodiments, clock circuit 56 may detect clock pulses as the point at which the phase modulation on the RF signal occurs and framing events encoded as relatively long time intervals with no clock pulses.

Thus, when a frame event is detected (i.e., a relatively long period of time without a change in voltage from the diodes 52 or an RF signal modulation), the clock circuit 56 may reset the sequence generator 58 to its initial state. The clock pulses following the frame event control timing for generation of the code sequence by the sequence generator 58, represented as g₁(i), which is preprogrammed in each marker 40.

Turning to FIG. 7A, an example of periodic code sequences 700a of length N=8 is shown that can be used for code multiplexing of four reflector markers triggered using RFID signal modulation. In this example, the probe 20 transmits a frame 702 including eight clock pulses having predetermined time lengths using RF signal modulation. The frame 702 is separated from other frames by a relatively long period of with no RF signal modulation. Between the frames, the power harvesting block may be configured to harvest electrical energy from the diodes or RFID antenna). As can be seen, the first marker (labeled Tag 1) includes a sequence generator that has a code sequence configured to alternately open and close the switch of the first marker with each clock pulse, while the second marker (labeled Tag 2) has a code sequence that opens and closes the switch with every other pulse. In this example, the four markers modulate their reflective properties in a different manner than each other, which the processor(s) of the probe 20 and/or display unit 30 may process to identify and/or locate each of the markers 40.

Similarly, FIG. 7B illustrates an example of periodic code sequences 700b of length N=8 is shown that can be used for code multiplexing of four reflector markers triggered using IR light. In this example, the probe 20 transmits a frame 704 including eight clock pulses having predetermined time lengths using IR light pulses. In the illustrated embodiment, the frame 702 is separated from other frames by a relatively long period of with no light pulses and the IR light left on. Between the frames, the power harvesting block may be configured to harvest electrical energy from the diodes or RFID antenna). As can be seen, the first marker (labeled Tag 1) includes a sequence generator that has a code sequence configured to alternately open and close the switch of the first marker with each clock pulse, while the second marker (labeled Tag 2) has a code sequence that opens and closes the switch with every other pulse. In this example, the four markers modulate their reflective properties in a different manner than each other, which the processor(s) of the probe 20 and/or display unit 30 may process to identify and/or locate each of the markers 40.

The processor(s) of the probe 20 and/or display unit 30 may perform separation and analysis of waveforms associated with individual reflectors using the orthogonal code sequences and the exemplary algorithm described below. To describe a method for the use of orthogonal sequences we consider a set of sequences in the form of s₁ (i)={−1,1}, instead of g₁(i)={0,1}, where index i=0 . . . N−1. These sequences contain the same and even number of symbols N=2m. They are balanced and orthogonal, i.e.,

$${\sum }_{i = 0}^{N - 1} {s}_{l} ( i ) = 0 and {\sum }_{i = 0}^{N - 1} {s}_{l} ( i ) {s}_{k} ( i ) = \{ \begin{matrix}N , l = k \\ 0 , l \neq k\end{matrix}$$

Modulation of the RF signal measured in RFID reader includes the backscatter signals received from each RFID tag with index k and for the code sequences sₖ(i) can be written using complex amplitudes as

$$A ( i ) = {A}_{S} + {\sum }_{k = 1}^{K} {s}_{k} ( i ) {a}_{k} + {W}_{Noise} ( i )$$

where A_{S} is the average amplitude of the received signal in RFID reader and aₖ is the effect of signal modulation in the reader caused by the switching of antenna coil in the k-th RFID tag.

Detection and localization of tags can be done by evaluation of their modulation amplitudes aₖ. The amplitude of a specific tag can be calculated from the total modulation of the received signal A(i) by locking it to the corresponding code sequence sₖ(i). It can be achieved by multiplying the received sequence A(i) with the corresponding code symbol sₖ(i) and calculating the sum of the results for the complete number of symbols in the sequence. i.e. N. The result of such multiplication and summation

$${R}_{tag} ( l ) = {\sum }_{i = 0}^{N - 1} {s}_{l} ( i ) A ( i ) ,$$

can be unfolded by substituting the received sequence A(i) with its components, and written as follows

$${R}_{tag} ( l ) = {\sum }_{i = 0}^{N - 1} {s}_{l} ( i ) {A}_{S} + {\sum }_{i = 0}^{N - 1} [ {s}_{l} ( i ) {\sum }_{k = 1}^{K} {s}_{k} ( i ) {a}_{k} ] + {\sum }_{i = 0}^{N - 1} {s}_{l} ( i ) {W}_{Noise} ( i ) .$$

The equation for R_{tag}(l) is a sum three terms. The first one gives zero due to balance property of code sequence. Indeed,

$${\sum }_{i = 0}^{N - 1} {s}_{l} ( i ) {A}_{S} = {A}_{S} {\sum }_{i = 0}^{N - 1} {s}_{l} ( i ) = 0.$$

The second term can be rewritten as

$${a}_{l} {\sum }_{i = 0}^{N - 1} {s}_{l} ( i ) {s}_{l} ( i ) + {\sum }_{{k = 1}_{k \neq l}}^{K} {a}_{k} {\sum }_{{i = 0}_{k \neq l}}^{N - 1} {s}_{l} ( i ) {s}_{k} ( i ) = {Na}_{l} .$$

Therefore, the result of the multiplication and summation, R_{tag}(l), is

$${R}_{tag} ( l ) = {Na}_{l} + {\sum }_{i = 0}^{N - 1} {s}_{l} ( i ) {W}_{Noise} ( i )$$

and does not depend on the amplitudes of the other RFID tags because their contribution to R_{tag}(l) equals to zero due to orthogonal properties and balanced selection of sequences. Computing the modulation amplitude for each tag one can estimate the proximity of the tag by one of the known method, see for example U.S. Pat. No. 10,499,832.

To obtain waveforms of the modulation of the other markers, the processor(s) may perform the same processing, i.e., repeated using the code sequences preprogrammed in the respective markers. The sets of orthogonal sequences may be designed by utilizing a periodic sequence, such as that shown in FIG. 7A and FIG. 7B and described above, or using other methods.

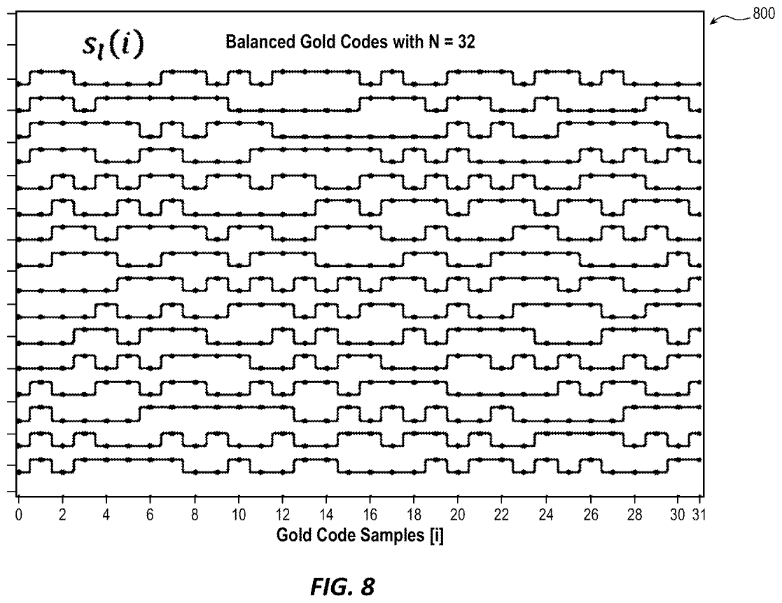

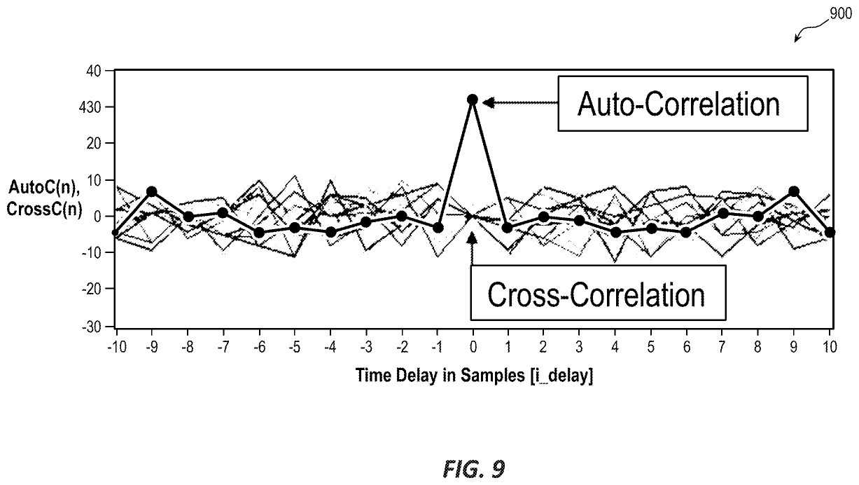

For example, FIG. 8 shows another exemplary embodiment using Gold Code sequences 800 specially conditioned to support properties of balance and orthogonality. These sequences use a Gold Code algorithm to generate a set of sequences of length thirty two (32) symbols, modified to support the balance property by adding an extra symbol at the beginning of each sequence. As a result, the cross-correlation Σᵢ₌₀^{N-1}sₖ(i)sₖ(i+i_{delay}) between each two sequences has zero value as shown in FIG. 9 (see i_{delay}=0).

With the reflected signals separated for each marker, the processor(s) may then process the individual signals to locate the individual markers, i.e., process the separated signals to determine a distance from the probe 20 to the respective markers 40. This processing may be performed substantially simultaneously, allowing information regarding each of the markers 40 to be presented to the user at the same time, e.g., on the display 34 of the display unit 30.

For example, each individual signal associated with a marker may be processed initially to identify the amplitude (or power envelope) of the signal waveform, and then determine the time delay of the return pulse in the signal to locate the marker. For example, to provide a distance measurement, time delay of the returned pulse may be measured with respect to the time of cross talk pulse, associated with a reflection from the probe antenna interfacing the tissue, to evaluate propagation delay in the path, e.g., from the probe 20 to the marker 40 and back to the probe 20, e.g., as shown in FIG. 1, and, then the distance between the tip of the probe 20 and the marker 40 may be calculated taking into account the propagation speed of the ultrawide band pulse in tissue.

Alternatively, Gold Code sequences may be used in a continuous wave RF system, such as those disclosed in U.S. Ser. No. 10/499,832, where amplitude and phase shift of the separated signals characterizing the propagation time and attenuation of the CW signal in the tissue on the path from the probe 20 to the marker 40 and back to the probe may be used to identify and locate each marker.

It will be appreciated that the multiplexing processing, e.g., code division processing, described herein may be used with other RF systems and/or other medical or non-medical applications using RFID.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

In the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment.

It will be appreciated that various features are sometimes grouped together in a single embodiment, figure, or description thereof for the purpose of streamlining the disclosure. Many of these features may be used alone and/or in combination with one another.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest to the practitioner during use.

Recitation in the claims of the term “first” with respect to a feature or element does not necessarily imply the existence of a second or additional such feature or element.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

The embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A system for localization of a target tissue region within a patient's body, comprising:
a probe comprising one or more antennas for transmitting radiofrequency signals into a patient's body and receiving backscatter signals; and
a plurality of radio-frequency identification (RFID) markers sized for implantation within the patient's body, each marker comprising:
a switch;
an antenna coupled with the switch; and
a sequence generator, the sequence generator coupled to the switch to open and close the switch to modulate the backscatter signals based on the code sequence,
wherein the code sequences generated by the sequence generators of the plurality of markers modulate the backscatter signals in non-interfering patterns.

2. The system of claim 1, wherein the code sequences are orthogonal to one another.

3. The system of claim 1, wherein the probe uses the modulation of the backscatter signals to identify each of the plurality of markers.

4. The system of claim 1, wherein the probe uses amplitude of the backscatter signals to determine a distance between the probe and the marker.

5. The system of claim 1, wherein each RFID marker further comprises:
an energy converter configured for generating electrical energy; and
a power harvesting block coupled to the energy converter to generate electrical energy to operate electrical components of the marker.

6. The system of claim 5, wherein the energy converter is a coil antenna that converts the radiofrequency signals.

7. The system of claim 1, the probe further comprising a light source to transmit optical signals into the patient's body, wherein the optical signals transmitted by the probe comprises infrared light, and wherein the RFID marker comprises one or more photodiodes configured to convert the infrared light into electrical energy to power the sequence generator to the switch to open and close the switch to modulate the backscatter signals based on the code sequence.

8. The system of claim 1, wherein each RFID marker further comprises a clock circuit, wherein the clock circuit of each RFID marker is configured to identify a beginning of each frame, whereupon the clock circuit resets the sequence generator of the respective marker to generate the respective code sequence to open and close the switch a predetermined number of times such that the modulation of the backscatter signals from the plurality of markers is orthogonal and balanced.

9. The system of claim 8, wherein the clock circuit is further to detect clock pulses of each frame, wherein the clock pulses are indicated by the probe with phase modulations in the radiofrequency signals.

10. The system of claim 8, wherein the clock circuit is further to detect clock pulses of each frame, wherein the clock pulses are indicated by the probe with light pulses.

11. The system of claim 1, further comprising a processor configured to separate the backscatter signals from the plurality of RFID markers based, at least in part, on the code sequences to identify each of the plurality of markers substantially simultaneously.

12. The system of claim 11, wherein the processor is further configured to process separated individual waveforms to identify amplitude of the separated individual waveforms to provide an output corresponding to distances from the probe the respective RFID markers.

13. The system of claim 11, wherein the code sequences of the markers are balanced, and wherein the processor sums the results based on the orthogonal and balanced code sequences to provide the modulation of the separate individual waveforms of the respective RFID markers.

14. The system of claim 1, further comprising a display configured to provide an output corresponding to a distance between the probe and each of the plurality of RFID markers.

15. A probe for identifying and locating a plurality of markers implanted within a patient's body, comprising:
one or more antennas for transmitting radio frequency signals into a patient's body and receiving backscatter signals from a plurality of RFID markers in the patient's body, wherein the plurality of markers modulate the backscatter signals based on orthogonal code sequences; and
a processor for processing the backscatter signals to separate the modulated signals from the plurality of RFID markers based at least in part on the orthogonal code sequences to identify each of the plurality of RFID markers substantially simultaneously.

16. The probe of claim 15, wherein the processor is further configured to process separated individual waveforms to identify amplitude of the separated individual waveforms to provide an output corresponding to distances from the probe the respective markers.

17. The probe of claim 15, wherein the probe provides a clock signal to the plurality of RFID using modulations in the radiofrequency signals.

18. The probe of claim 15, wherein the probe provides a clock signal to the plurality of RFID using light pulses.

19. A plurality of RFID markers sized for implantation within a patient's body, each marker comprising:
one or more antennas for receiving radio frequency and generating power from the received radio frequency;
a power harvesting block coupled to the one or more antennas
a clock circuit coupled to the power harvesting block;
a switch coupled in parallel to the one or more antennas; and
a sequence generator coupled to the clock circuit to generate a code sequence, the sequence generator coupled to the switch to open and close the switch to modulate electromagnetic backscatter signals transmitted by the marker based on the code sequence, wherein the code sequence generated by the sequence generator is a pattern that does not interfere with code sequences associated with a plurality of other RFID markers.

20. The plurality of RFID markers of claim 19, wherein the code sequences are orthogonal.
